(19) United States
(12) Patent Application Publication
(10) Pub. No.: US 2023/0219881 A1
(43) Pub. Date: Jul. 13, 2023

(54) Synthesis of Spermidine, Spermine, and Free Bases Thereof

(71) Applicants: Compound Solutions Inc., Carlsbad, CA (US); Shaoxing King-Year Biochemical Co. Ltd., Zhejiang (CN)

(72) Inventors: Matthew Titlow, Carlsbad, CA (US); Xinglong Gu, Zhejiang (CN)

(73) Assignees: Compound Solutions Inc., Carlsbad, CA (US); Shaoxing King-Year Biochemical Co., Ltd., Zhejiang (CN)

(21) Appl. No.: 18/085,157

(22) Filed: Dec. 20, 2022

Related U.S. Application Data
(60) Provisional application No. 63/298,563, filed on Jan. 11, 2022.

(51) Int. Cl. C07C 209/62

(52) U.S. Cl. CPC C07C 209/62

(57) ABSTRACT

A simplified synthetic process for production of spermine and spermidine are presented that can provide spermine and spermidine in free base or salt form. Advantageously, the synthetic procedure is safe, simple, and environmentally friendly, while at the same time also being cost-effective.

This application claims priority to copending U.S. provisional application 63/298,563, which was filed Jan. 11, 2022, and which is incorporated by reference herein.

### FIELD OF THE INVENTION

The field of the invention is synthesis of polyamines, and especially as it relates to synthesis of spermidine hydrochloride and its free base.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Spermidine is a natural polyamine compound, which widely exists in animals, plants, marine plankton, and algae. In recent years, it has been reported that spermidine participates in many physiological activities of organisms and plays an important role in the growth and proliferation of animal and plant cells. Indeed, spermidine shows a variety of desirable physiological actions in human, including an antioxidant effect, anti-aging effects, antidepressant effects, analgesia, and lowering blood pressure. At the same time, spermidine is also an important pharmaceutical intermediate, which is used in the synthesis of certain active pharmaceutical ingredients. Unfortunately, while spermidine and spermidine are found in various plant and plant extracts (e.g., black Lycium barbarum, safflower, tea, etc.), their content is typically low, and extraction is often difficult.

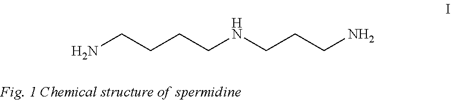

CN109096122 discloses a method for preparing spermidine from 1-aminopropanol and butyrolactone through aminolysis, reduction, amino protection, substitution and deprotection steps. The method in the '122 patent uses lithium aluminum tetrahydride as reducing agent, which is expensive and poses potential safety hazards during industrial production, and the synthetic path of the '122 patent is shown in Scheme 1 below. Moreover, the introduction of an amino group with phthalimide is of relatively low efficiency and generates large amounts of waste, rendering such synthetic process unsuitable for large-scale production.

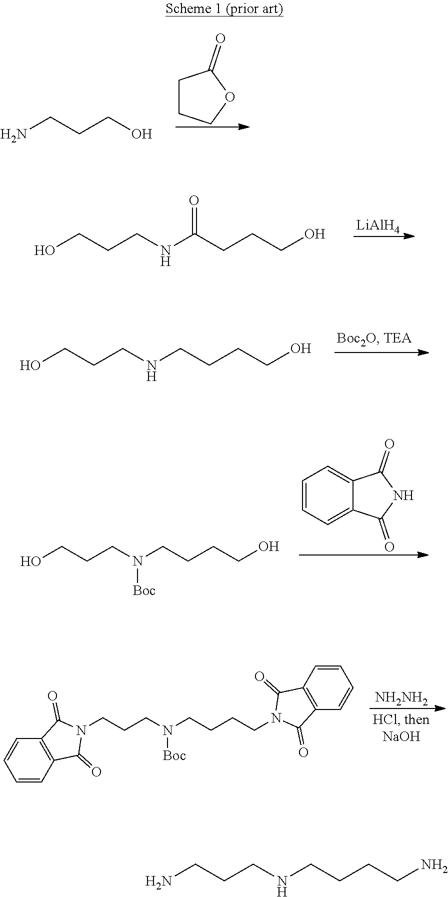

Thus, even though various methods for isolation and synthesis of spermidine are known in the art, all or almost all of them suffer from several drawbacks. Therefore, there remains a need for improved methods of synthesis of spermidine that are safe, simple, environmentally friendly, and cost effective.

### SUMMARY OF THE INVENTION

The inventive subject matter is directed to various methods of preparing spermine and/or spermidine in free base or salt form, and especially hydrochloride form. The methods presented herein are simple, environmentally safe, and cost-effective.

In one aspect of the inventive subject matter, the inventors contemplate a method of producing spermidine hydrochloride that includes a step of reacting 1,4-diaminobutane with a protecting group to thereby produce a partially protected diaminobutane that has a single reactive amino group, and a further step of reacting the single reactive amino group of the partially protected diaminobutane with acrylonitrile to thereby produce a protected cyano intermediate. In yet another step, the protected cyano intermediate is subjected to hydrogenation to thereby produce a partially protected spermidine, and in a still further step, the protecting group is removed from the partially protected spermidine to thereby produce the spermidine hydrochloride.

Most typically, but not necessarily, the protecting group is di-tert-butyl decarbonate, and/or the hydrogenation is a catalytic hydrogenation. It is still further contemplated that the protecting group is removed using hydrochloric acid. Where desired, contemplated methods may also include an additional step of alkalinizing the spermidine hydrochloride (e.g., using a carbonate, a hydroxide, or a methoxide). In still further contemplated methods, the spermidine may be subjected to enzymatic catalysis with spermine synthase to thereby produce spermine.

In another aspect of the inventive subject matter, the inventors contemplate a method of producing spermine hydrochloride that includes a step of reacting 1,4-diaminobutane with acrylonitrile to thereby produce a bis-cyano intermediate and a further step of subjecting the bis-cyano intermediate to hydrogenation (e.g., catalytic hydrogenation) to thereby produce a spermine. In such methods, the spermine can be produced as a spermine hydrochloride salt when the step of hydrogenation is performed in the presence of hydrochloric acid. Where desired, contemplated methods may include an additional step of subjecting the spermine to enzymatic catalysis with spermine synthase to thereby produce spermidine.

Various objects, features, aspects, and advantages of the inventive subject matter will become more apparent from the following detailed description of preferred embodiments.

### DETAILED DESCRIPTION

In the present disclosure, and unless otherwise specified, the scientific and technical terms used have the meaning generally understood by those skilled in the art. Moreover, the laboratory operational steps used in this disclosure are conventional steps widely used in the corresponding fields. In addition, and unless otherwise specified, the raw materials or reagents used in this disclosure are commercially available. All reagents are commercial grade and used according to the common standards.

The inventors have now discovered a conceptually simple and effective method of synthesizing spermidine that follows the exemplary and general 4-step reaction sequence as is shown in Scheme 2 below:

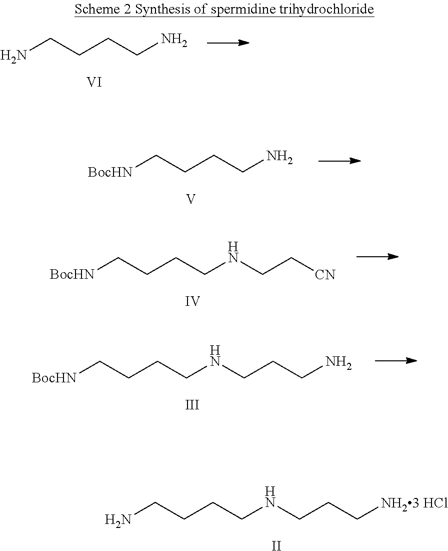

In general, 1,4-butanediamine (VI) is reacted with di-tert-butyl dicarbonate in a first solvent at a certain temperature to produce 4-(Boc) butylamine (V), which is then reacted with acrylonitrile in a second solvent to produce the protected amine compound (IV). Compound (IV) is then hydrogenated in a third solvent in the presence of a catalyst under certain pressure to produce protected compound (III), which is subsequently reacted in a fourth solvent in the presence of hydrochloric acid or hydrogen chloride to afford spermidine trihydrochloride (II).

In this context, it should be appreciated that following substantially the same general chemical process, spermine can also be synthesized. More specifically, in such process protection of one amino group in the 1,4-butanediamine (VI) is not required, and reaction with acrylonitrile will lead to a product (N,N′-Bis-(2-cyanoethyl)-1,4-diaminobutane) in which each of the nitrogen atoms is covalently bound to a cyanoethyl group. The so obtained product can then be subjected to hydrogenation and reaction with HCl as shown above to so yield spermine tetrahydrochloride.

In some examples of the inventive subject matter, the first solvent involved in step (1) is an organic solvent, water, or a mixed solvent of organic solvent and water. Most preferably, the organic solvent is selected from one or several of dioxane, tetrahydrofuran, and water.

In some examples of the inventive subject matter, the molar ratio of 1,4-butanediamine (VI) to di-tert-butyldicarbonate in step (1) is 2.5˜4.5:1.0. In some examples of the inventive subject matter, the reaction temperature in step (1) is −10˜50° C.; Preferably, the reaction temperature is −10˜30° C.

In some examples of the inventive subject matter, the second solvent involved in step (2) is an organic solvent, and preferably an alcohol or ketone solvent (e.g., methanol, ethanol, and/or acetone). In some examples of the inventive subject matter, the reaction temperature in step (2) is −10˜50° C. In some examples of the inventive subject matter, the molar ratio of compound (V) to acrylonitrile in step (2) is 1.0:1.0˜2.0.

In some examples of the inventive subject matter, the third solvent involved in step (3) is an organic solvent, and preferably an alcoholic solvent such as methanol and/or ethanol.

In some examples of the inventive subject matter, the catalyst described in step (3) is palladium acetate, palladium carbon, palladium carbon hydroxide, platinum carbon or Raney nickel. Preferably, but not necessarily, the catalyst is palladium acetate. When Raney nickel is selected as the catalyst, the mass ratio of catalyst to compound (IV) is typically 0.2˜1.0:1.0. When the catalyst is palladium acetate, the mass ratio of palladium acetate to compound (IV) is typically 1:100˜200. When the catalyst is palladium carbon, palladium hydroxide carbon or platinum carbon, the mass ratio of palladium, palladium hydroxide or platinum to compound (IV) in the catalyst is typically 1:100˜200. In some examples of the inventive subject matter, the hydrogenation pressure of step (3) is 2.0˜5.0 MPa. The reaction temperature is typically between 30˜120° C., and preferably between 60˜120° C.

In some examples of the invention, the fourth solvent involved in step (4) is an organic solvent, and preferably an alcohol, an ester, and/or an ether solvent. For example, suitable solvents include methanol, ethanol, ethyl acetate, and/or tetrahydrofuran. In some examples of the invention, the reaction temperature of step (4) is 0˜80° C.

As will be readily appreciated, spermidine HCl can be readily converted to the corresponding free base spermidine in a simple and cost-effective manner that provides a product of high quality. To that end, a synthetic method of spermidine can be carried out as shown in the exemplary step as shown in Scheme 3:

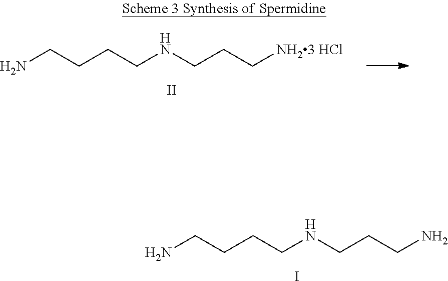

Compound (II) as obtained above is alkalinized in a fifth solvent in the presence of a base to so form spermidine (I). In some examples of the inventive subject matter, the fifth solvent is one or several organic solvents or water. Preferably, the organic solvent is an alcohol or mixture of alcohols such as methanol, ethanol, and/or isopropanol.

In some examples of the invention, the base is one or several of inorganic base or alcoholic alkali metal salt. For example, the inorganic base may be selected from one or several of potassium carbonate, sodium hydroxide, potassium hydroxide, and lithium hydroxide. Suitable alkoxide alkali metal salt include one or several of sodium methoxide, sodium tert-butoxide or potassium tert-butoxide. The molar ratio of the base to compound (II) is 3˜6:1, and in some examples of the invention, the reaction temperature is 0˜70° C.

Thus, it should be appreciated that the route of synthesis uses commercially available 1,4-butanediamine as a raw material, protects an amino group with tert-butoxycarbonyl and reacts with acrylonitrile, the intermediate is hydrogenated and deprotected to provide spermidine trihydrochloride, and the high-purity spermidine can be obtained from spermidine trihydrochloride by simple distillation after alkalinization. The preparation of spermidine trihydrochloride has only four steps, which is safe, simple, green, and cost effective. The process only involves two separation and purification steps. Preferably, the process operation yield is ≥68%. After simple distillation, the purity of spermidine obtained from spermidine trihydrochloride by this route can reach ≥99.5%, meeting the quality requirements of food and medicine.

Likewise, where spermine is desired, the route of synthesis uses commercially available 1,4-butanediamine as a raw material and reacts the 1,4-butanediamine with acrylonitrile. The resultant product is hydrogenated to provide spermine tetrahydrochloride, and the high-purity spermine can be obtained from spermine tetrahydrochloride by simple distillation after alkalinization. The preparation of spermine tetrahydrochloride has only three steps, which is safe, simple, green, and cost effective. The process only involves two separation and purification steps. Preferably, the process operation yield is ≥68%. After simple distillation, the purity of spermine obtained from spermidine tetrahydrochloride by this route can reach ≥99.5%, meeting the quality requirements of food and medicine.

Alternatively, it should be noted that spermine can also be obtained from spermidine using spermine synthase as biocatalyst. Likewise, it is contemplated that putrescine can be obtained from spermidine using spermidine synthase as biocatalyst.

It should be further appreciated that the above synthetic route solves several problems ordinarily associated with spermidine free base from a variety of sources (natural and synthetic). Pure spermidine base is typically highly viscous and tacky without any appreciable flowability rendering pure spermidine very difficult to process and/or dispense. Moreover, spermidine also rapidly oxidizes and light and oxygen must be avoided to maintain product quality. To circumvent such issues, spermidine can be micro-encapsulated or formulated in an oil suspension. However, spermidine is often not easily oil dispersible. In contrast, the spermidine hydrochloride produced by the present disclosure is formed as a stable powder that can be readily dispensed and handled. Moreover, spermidine hydrochloride is also relatively resistant to oxidation and is readily water soluble. As will be readily appreciated, where spermidine free base is produced from spermidine hydrochloride as presented herein, the product is readily water soluble and can be handled without significant delay from its time of production, thereby reducing issues associated with hygroscopicity and oxidative damage. Similar benefits also apply to the spermine product and production process.

With respect to contemplated uses, it should be appreciated that the spermine and the spermidine (as HCl salts or as free base) can be used alone or in combination in a variety of formulations, and especially preferred formulations are nutritionally acceptable formulations in solid and liquid form. For example, spermine and/or spermidine can be formulated into tablets, capsules, or other known formats suitable for use with nutritional supplements, as well as into powders (ready-to-use or for mixing with a liquid), snack bars, baked goods, etc. Likewise, spermine and/or spermidine can be formulated into a beverage that may be used directly by a consumer or as a concentrate for dilution with or addition to another beverage.

### EXAMPLES

### Example 1: Synthesis of Spermidine Trihydrochloride

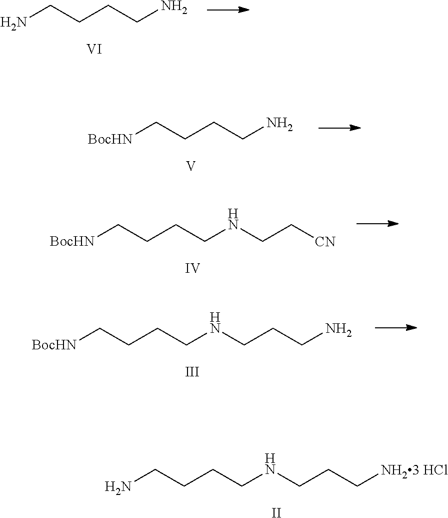

Step 1: Dissolve compound (VI) (0.1 mol) in dioxane (2 L) and add di-tert-butyl dicarbonate (0.33 mol) at 20˜30° C. After addition, the reaction is maintained at 20-30° C. for 12 hours, and TLC showed that the basic reaction is complete. After concentration, it is dissolved with dichloromethane and washed with water. The concentrated compound (V) is obtained and directly used in the next step. The HPLC shows that di-tert-butoxycarbonyl protection: mono-tert-butyl dicarbonate protection=15:85.

Step 2: The compound (V) (calculated as 0.33 mol) obtained in the previous step is dissolved in methanol (1 L), and freshly distilled acrylonitrile (0.4 mol) is added at 20˜30° C. After addition, the reaction is carried out at 40˜50° C. for 6 hours, and TLC showed that the compound (V) is basically consumed completely. After concentration, compound (IV) is obtained by vacuum distillation (0.25 mol, the total yield of two steps is 75%).

Step 3: Dissolve compound (IV) (0.25 mol) in methanol, bubble ammonia gas into the solution to saturation at 20˜30° C., and add palladium acetate (0.6 g, mass of palladium acetate/mass of compound (IV)=1/100). After hydrogenation for 24 hours at 60˜80° C. and 3˜3.5 MPa, TLC showed that compound (IV) is completely consumed. After filtration and concentration, the obtained compound (III) is directly used in the next step.

Step 4: Compound (III) (calculated as 0.25 mol) is dissolved in methanol (2 L), and hydrogen chloride gas is introduced at 20˜30° C. to saturation. After passing, continue stirring at 20˜30° C. for 6 hours. After concentration, add ethanol (1 L), re-slurry at 0˜10° C. and filter to obtain white solid (II) (0.22 mol, two-step yield 88%, purity 98.0%).

### Examples 2-10: Alternate Syntheses of Spermidine Trihydrochloride

Example 2: In step 1, tetrahydrofuran is selected as the solvent, and the molar ratio of di-tert-butyldicarbonate to compound (VI) is 4.5:1. Other operations are the same as step 1 of example 1. The solvent in step 2 is ethanol, and the equivalence ratio of acrylonitrile to compound (V) is 1.0:1.0. The other operations are the same as step 2 of example 1. The total yield of the first two steps is 78%. In step 3, palladium hydroxide carbon (20%) is selected as the catalyst, the mass ratio of catalyst to compound (IV) is 1.0:20, ethanol is selected as the solvent, the pressure is 2˜2.5 MPa and the temperature is 80˜100° C. The other operations are the same as step 3 of example 1; The operation of step 4 is the same as that of example 1. The yield of the two steps is 83% and the purity is 98.5%.

Example 3: In step 1, the solvent is water, and the molar ratio of di-tert-butyl dicarbonate to compound (VI) is 2.5:1.0. Other operations are the same as step 1 of example 1. The solvent in step 2 is acetone, and the molar ratio of acrylonitrile to compound (V) is 1.1:1.0. The other operations are the same as step 2 of example 1. The total yield of the first two steps is 70%. In step 3, palladium hydroxide carbon (20%) is selected as the catalyst, and the mass ratio of catalyst to compound (IV) is 1.0:10. The other operations are the same as step 3 in example 1. The operation of step 4 is the same as that of step 4 of example 1. The yield of the two steps is 80% and the purity is 98.5%.

Example 4: Step 1 is the same as step 1 of example 1. The solvent of step 2 is acetone, the molar ratio of acrylonitrile to compound (V) is 2.0:1.0, and the reaction temperature is −10˜5° C. The other operations are the same as step 2 of example 1. The total yield of the first two steps is 78%. In step 3, palladium carbon (10%) is selected as the catalyst, the mass ratio of catalyst to compound (IV) is 1.0:10, the solvent is dioxane, the temperature is 110˜120° C. and the pressure is 4.5˜5.0 MPa. The other operations are the same as step 3 of example 1. The operation of step 4 is the same as that of step 4 of example 1. The yield of the final two steps is 78% and the purity is 97.5%.

Example 5: Step 1 is the same as step 1 of example 1. The solvent in step 2 is methanol, the molar ratio of acrylonitrile to compound (V) is 1.2:1.0, and the reaction temperature is 10˜20° C. The total yield of the first two steps is 70%. In step 3, palladium acetate is selected as the catalyst, and the mass ratio of catalyst to compound (IV) is 1.0:200. The other operations are the same as step 3 of example 1. The operation of step 4 is the same as that of step 4 of example 1, the yield of the two steps is 85%, and the purity of the product spermidine trihydrochloride is 98.5%.

Example 6: The solvent in step 1 is dioxane/water (0.1 mol 1,4-butanediamine is dissolved in 1 L water and 1 L dioxane mixed solvent), and the other operations are the same as those in step 1 of example 1. Step 2 is the same as step 2 of example 1, and the total yield of the first two steps is 76%. In step 3, palladium acetate is selected as the catalyst, the mass ratio of catalyst to compound (IV) is 1.0:100, and the reaction temperature is 30˜40° C. The other operations are the same as in step 3 of example 1. The operation of step 4 is the same as that of step 4 of example 1. The yield of the two steps is 75% and the purity of the product spermidine trihydrochloride is 98.5%.

Example 7: The temperature of step 1 is 40˜50° C., and the other operations are the same as step 1 of example 1. HPLC shows that the protection product of di-tert-butoxycarbonyl: the protection product of mono-tert-butoxycarbonyl=23:77. Step 2 in example 7 are the same as step 2 in example 1, and the yield of the two steps is 66%. In step 3, platinum carbon (10%) is used as the catalyst, and the mass ratio of catalyst to compound (IV) is 1.0:200. The other operations are the same as step 3 of example 1. In step 4, ethyl acetate is used as the solvent, 6 mol/L hydrochloric acid is added, the mass of hydrochloric acid is 3 times the mass of compound (III), and the reaction temperature is 80° C. After the reaction, the ethyl acetate phase is separated, concentrated and beaten with ethanol. The other operations are the same as step 4 of example 1, the yield of the two steps is 80%, and the purity of the product spermidine trihydrochloride is 98.4%.

Example 8: Steps 1 to 3 are the same as steps 1 to 3 in example 1. In step 4, tetrahydrofuran is selected as the solvent, the reaction temperature is 0˜10° C. and saturated hydrogen chloride in methanol is added. The mass ratio of the saturated hydrogen chloride in methanol to compound (III) is 5.0:1.0. The other operations are the same as step 4 of example 1. The yield of the last two steps is 87%, and the purity of the product spermidine trihydrochloride is 98.4%.

Example 9: The operations of steps 1 and 2 are the same as steps 1 and 2 in example 1. In step 3, Raney nickel is selected as the catalyst, and the mass ratio of Raney nickel to compound (IV) is 0.2:1.0. Other operations are the same as step 3 in example 1, and the hydrogenation reaction is completed in about 36 hours. Step 4 is the same as step 4 in example 1. The yield of steps 3 and 4 is 80%, and the purity of the product spermidine trihydrochloride is 98.0%.

Example 10: The mass ratio of Raney nickel to compound (IV) in step 3 is 1.0:1.0. Other operations are the same as those in Example 9. The hydrogenation reaction is completed in about 18 hours. The yield of steps 3 and 4 is 81%, and the purity of spermidine trihydrochloride is 98.1%.

### Example 11: Synthesis of Spermidine

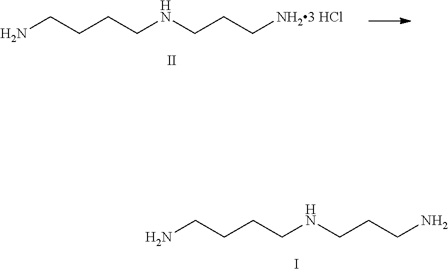

The Compound (II) (0.2 mol) is dissolved in methanol (1 L), potassium carbonate (0.6 mol) is added in several times, stirred at 20-30° C. for 4-6 hours, filtered, concentrated and distilled under reduced pressure (external temperature: 150-180° C., pressure: 15-20 Pa, fraction: 125-128° C.) to obtain colorless liquid compound (I) (0.17 mol, yield 85%) in 99.5% purity.

### Examples 12-14: Alternate Syntheses of Spermidine

Example 12: The solvent is ethanol, the base is sodium hydroxide, the molar ratio of sodium hydroxide to compound (II) is 3.5:1.0, and the reaction temperature is 0˜20° C. Other operations are the same as those in example 11. Spermidine is provided in 88% yield and 99.6% purity.

Example 13: The solvent is isopropanol, the base is sodium methoxide, the molar ratio of sodium methoxide to compound (II) is 4.0:1.0, and the reaction temperature is 50˜70° C. The other operations are the same as those in Example 11. Spermidine is provided in 90% yield and 99.5% purity.

The solvent is water, the base is lithium hydroxide, the molar ratio of lithium hydroxide to compound (II) is 6.0:1.0, and the reaction temperature is 30˜40° C. After the completion of reaction, it is concentrated, re-slurried in ethanol and filtered. After concentration, it is distilled under reduced pressure. The yield is 80% and the purity is 99.5%.

Comparative case 1: When acetone is selected as the solvent in step 1, the other operations are the same as in step 1 of the example 1. The HPLC shows that the protection product of di-tert-butoxycarbonyl: mono-tert-butoxycarbonyl=41:59.

Comparative case 2: When the hydrogen pressure in step 3 is 2.0 MPa, the other operations are the same as those in step 3 of example 1. After 48 hours of reaction, TLC shows that about 80% of raw materials are left.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A method of producing spermidine hydrochloride, comprising:
reacting 1,4-diaminobutane with a protecting group to thereby produce a partially protected diaminobutane that has a single reactive amino group;
reacting the single reactive amino group of the partially protected diaminobutane with acrylonitrile to thereby produce a protected cyano intermediate;
subjecting the protected cyano intermediate to hydrogenation to thereby produce a partially protected spermidine; and
removing the protecting group from the partially protected spermidine to thereby produce the spermidine hydrochloride.

2. The method of claim 1, wherein the protecting group is di-tert-butyl dicarbonate.

3. The method of claim 1, wherein the hydrogenation is a catalytic hydrogenation.

4. The method of claim 1, wherein the protecting group is removed using hydrochloric acid.

5. The method of claim 1, further comprising a step of alkalinizing the spermidine hydrochloride to thereby form spermidine free base.

6. The method of claim 5, wherein the alkalinizing is performed using a carbonate, a hydroxide, or a methoxide.

7. The method of claim 1, further comprising a step of subjecting the spermidine to enzymatic catalysis with spermine synthase to thereby produce spermine.

8. A method of producing spermine hydrochloride, comprising:
reacting 1,4-diaminobutane with acrylonitrile to thereby produce a bis-cyano intermediate; and
subjecting the bis-cyano intermediate to hydrogenation to thereby produce a spermine;
wherein the spermine is a spermine hydrochloride salt when the step of hydrogenation is performed in the presence of hydrochloric acid.

9. The method of claim 7, wherein the hydrogenation is a catalytic hydrogenation.

10. The method of claim 8 or 9, further comprising a step of subjecting the spermine to enzymatic catalysis with spermine synthase to thereby produce spermidine.

11. A method of producing a nutritional supplement, comprising:
producing spermine and/or spermidine as a free base or as a hydrochloride salt using a method of claim 1 or claim 8; and
combining the spermine and/or spermidine as the free base or as the hydrochloride salt with a nutritionally acceptable carrier to so form the nutritional supplement.

12. The method of claim 12, wherein the supplement is formulated as a tablet, a capsule, a powder, or a drink.

13. The method of claim 12 wherein a unit dose of the supplement provides between 100 mcg and 50 mg of the spermidine as the free base or as the hydrochloride salt and/or between 10 mcg and 20 mg of the spermine as the free base or as the hydrochloride salt.
